(19) United States
(12) Patent Application Publication
(10) Pub. No.: US 2023/0329590 A1
(43) Pub. Date: Oct. 19, 2023

(54) Non-Contact Monitoring System and Method

(71) Applicant: Covidien LP, Mansfield, MA (US)

(72) Inventors: Christopher NELSON, Longmont, CO (US); Paul S. Addison, Carlsbad, CA (US); Dean MONTGOMERY, Edinburgh (GB)

(73) Assignee: Covidien LP, Mansfield, MA (US)

(21) Appl. No.: 18/296,208

(22) Filed: Apr. 5, 2023

Related U.S. Application Data
(60) Provisional application No. 63/331,115, filed on Apr. 14, 2022.

(51) Int. Cl. A61B 5/113; A61B 5/00

(52) U.S. Cl. CPC A61B 5/1135; A61B 5/742; A61B 5/0077

(57) ABSTRACT

Systems and methods for non-contact monitoring are disclosed herein. An example system includes at least one depth determining device configured to determine depth data representing depth across a field of view; a processor configured to process the depth data to obtain time varying depth or physiological information associated with respiration and/or another physiological function; and a projector configured to project one or more images into the field of view, wherein at least part of the one or more images is based on the obtained time varying depth or physiological information.

### CROSS-REFERENCE TO RELATED APPLICATIONS

This application claims the benefit of U.S. Provisional Application No. 63/331,115 filed Apr. 14, 2022, entitled “Non-Contact Monitoring System and Method,” which is incorporated herein by reference in its entirety.

### FIELD

The present invention relates to a system and method for non-contact monitoring of a subject.

### BACKGROUND

Video-based monitoring is a new field of patient monitoring that uses a remote video camera to detect physical attributes of a patient. This type of monitoring may also be called “non-contact” monitoring in reference to the remote video sensor, which does not contact the patient.

It is known to use depth sensing devices to determine a number of physiological and contextual parameters for patients including respiration rate, tidal volume, minute volume, effort to breathe, activity, presence in bed. It is also known to provide a visualization of breathing of the patient on a monitor screen.

### SUMMARY

In accordance with a first aspect, there is provided a system comprising: at least one depth determining device configured to determine depth data representing depth across a field of view; a processor configured to process the depth data to obtain time varying depth or physiological information associated with respiration and/or another physiological function; and a projector configured to project one or more images into the field of view, wherein at least part of the one or more images is based on the obtained time varying depth or physiological information.

The processor may be configured to process the depth data to generate image data representative of the sequence of projected images and the projector is configured to receive said image data. The projector may be configured to project the one or more images onto the skin and/or clothes and/or bedclothes of the subject.

The at least one depth determining device may comprise a depth sensing device configured to sense depth. The depth determining device may be configured to produce said depth data based on the sensed depth.

The depth data may be sensed from a region of interest of a subject thereby to obtain time varying depth or physiological information associated with respiration and/or another physiological function of the subject and wherein the image is projected back on to the region of interest of the subject.

The obtained time varying depth or physiological information may be obtained as a function of position across the field of view of the depth determining device.

The projected one or more images may be in spatial correspondence with the obtained time varying depth or physiological information.

The time varying depth or physiological information may comprise a sign and/or a magnitude of a calculated change in depth.

The time varying depth or physiological information may comprise a physiological parameter.

The physiological parameter may comprise at least one of: a respiration rate, pulse rate, tidal volume, minute volume, effort to breathe, oxygen saturation.

The time varying depth or physiological information may comprise a total displacement or velocity of a region of the subject over a breathing cycle.

The time varying depth or physiological information may comprise a magnitude and/or sign of movement relative to the depth determining device.

The one or more projected images may comprise one or more visual indicators having an appearance based at least in part on the determined time varying depth or physiological information.

The appearance of the one or more visual indicators may comprise at least one of: a colour, shade, pattern, concentration and/or intensity.

The depth data may be obtained for a region of interest of a subject and the visual indicator of the one or more projected images may substantially span the region of interest.

The one or more visual indicators may comprise at least one of: an overlay, a boundary and/or an area defining a region, textual or numerical data, an arrow, one or more contours.

The one or more visual indicators may comprise two or more visual indicators corresponding to two or more regions, wherein the appearance of each of the two or more visual indicators is based on the time varying depth information or physiological information obtained for that region.

The two of more images may comprise a sequence of moving images and are projected in real time such that the projected images change in response to the changes in the time varying depth or physiological information.

The one or more visual indicators may have a first appearance when the determined time varying depth information and/or physiological information is indicative of movement of the region away from the depth determining device and a second appearance when the determined time varying depth information corresponds to movement of the at least one region toward the depth determining device.

The at least one visual indicator may have a further appearance when the determined time varying depth information is indicative of lack of movement of the region relative to the depth determining device.

The processor may be configured to determine noise information associated with the time varying depth or physiological information and wherein the one or more images is based on the determined noise information. The processor may be configured to project and/or adjust one or more visual indicators based on the determined noise information.

The projector may be an optical projector configured to generate and project an optical image.

The at least one depth determining device may comprise at least one of: a depth sensing camera, a stereo camera, a camera cluster, a camera array, a motion sensor.

In accordance with a second aspect, that may be provided independently, there is provided a method comprising: determining depth data representing depth across a field of view;

processing the depth data to obtain time varying depth or physiological information associated with respiration and/or another physiological function; and projecting one or more images into the field of view, wherein at least part of the one or more images is based on the obtained time varying depth or physiological information.

In accordance with a third aspect, that may be provided independently, there is provided a non-transitory computer readable medium comprising instructions operable by a processor to: receive depth data representing depth across a field of view; process depth data to obtain time varying depth or physiological information associated with respiration and/or another physiological function; and generate projection image data representative of one or more images for projecting into the field of view, wherein at least part of the one or more images is based on the obtained time varying depth or physiological information

Features in one aspect may be provided as features in any other aspect as appropriate. For example, features of a method may be provided as features of a system and vice versa. Any feature or features in one aspect may be provided in combination with any suitable feature or features in any other aspect.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure. The drawings should not be taken to limit the disclosure to the specific embodiments depicted, but are for explanation and understanding only.

FIG. 1. is a schematic view of a patient monitoring system for monitoring a subject, the system having a depth determining device and a projector for projecting an image based on obtained time varying depth sensing information and/or physiological information, in accordance with various embodiments;

FIG. 2 is a block diagram that illustrates a patient monitoring system having a computing device, a server, one or more image capture devices, and a projector, and configured in accordance with various embodiments;

FIG. 3A is a rendered image of a patient monitoring system and support apparatus and FIG. 3B depicts the patient monitoring system and support apparatus adjacent to a hospital bed, in accordance with various embodiments;

FIGS. 4A and 4B are illustrations of images projected on to a subject, in accordance with an embodiment;

FIG. 5A and FIG. 5B are illustrations of an image projected on to a subject, in accordance with a further embodiment;

FIG. 6 is an illustration of an image projected on to a subject having a projected region and one or more further visual indicators, in accordance with a further embodiment.

### DETAILED DESCRIPTION

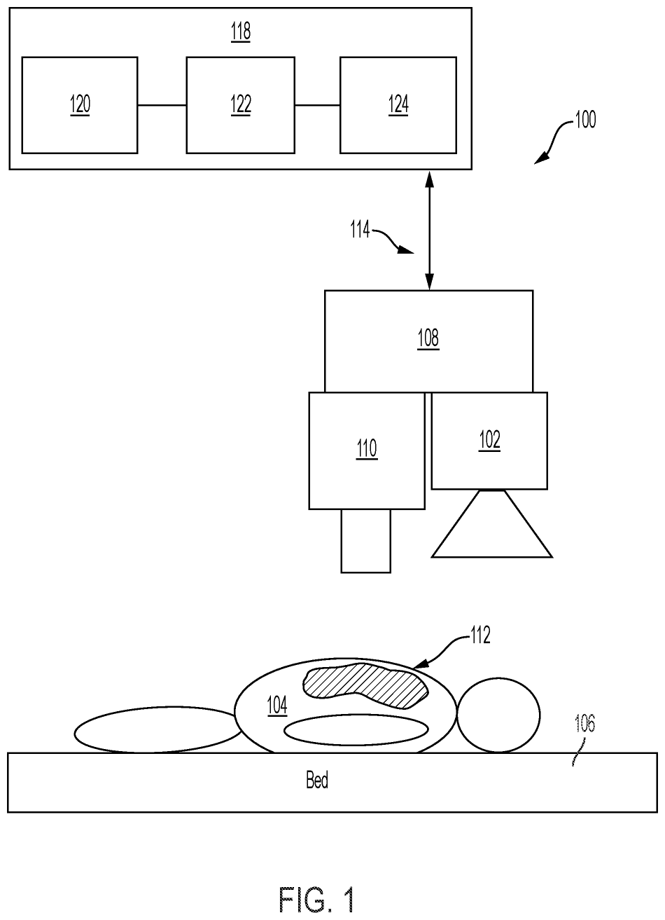

FIG. 1 is a schematic view of a patient monitoring system 100 for monitoring a subject, for example patient 104. The patient monitoring system 100 may be provided in a number of settings. In the present embodiment, the system 100 is described in the context of a clinical environment, for example, a hospital, and is provided adjacent to a hospital bed 106.

The system 100 includes a non-contact depth determining device, in particular, an image-based depth sensing device. In the present embodiment, the image-based depth sensing device is a depth-sensing camera 102. The camera 102 is at a remote position from the patient 104 on the bed 106. The camera 102 is remote from the patient 104, in that it is spaced apart from the patient 104 and does not contact the patient 104. In particular, the camera 102 is provided at an elevated position at a height above the bed and angled to have a field of view of at least part of the bed 106. It will be understood that, while FIG. 1 depicts a single image capture device (camera 102) the system may have more than one image capture or depth sensing devices.

In the embodiment of FIG. 1, the field of view of the camera 102 includes at least an upper portion of the bed 106 such that, in use, a torso or chest region of the patient 104 is visible in the field of view 116, to allow respiratory information to be obtained using obtained depth data.

The camera 102 generates a sequence of images over time. In the described embodiments, the camera 102 is a depth sensing camera, such as a Kinect camera from Microsoft Corp. (Redmond, Wash.) or a RealSense depth camera from Intel (Intel, Santa Clara, California). A depth sensing camera can detect a distance between the camera and objects in its field of view. This depth information can be used, as disclosed herein, to determine a region of interest (ROI) to monitor on the patient. Once a ROI is identified, that ROI can be monitored over time, and depth data associated with the region of interest obtained. The obtained depth data is representative of depth information as a function of position across the field of view of the camera.

The system also has a projector 110. The projector 110 is configured to project a sequence of images on to a region of the patient 104. In the present embodiment, the camera 102 is configured to obtain depth data for the region of interest of the patient (for example, the torso region) and the projector 110 is configured to project a sequence of images back on to the region of interest.

The projector 110 is an optical-based projector that projects images using visible light (for example, light in the wavelength range 400 nm to 700 nm). It will be understood that the projector 110 is configured to project light onto a surface. As described in the following, the projected images are based on processing of the depth data obtained by camera 102. In particular, as described in further detail in the following, the projected images include one or more visual indicators that are based on depth information or physiological information determined by the processing the depth data. In particular, the appearance and/or data conveyed by the one or more visual indicators is based on the depth information or physiological information obtained by processing the depth data.

The projector is configured to project one or more images onto a projection surface. In the following embodiments, the projection surface substantially corresponds to part of the patient (for example, the skin, clothes or upper bed sheet). It will be understood that the images are projected onto a moving surface. For example, the region of interest may be moving due to breathing.

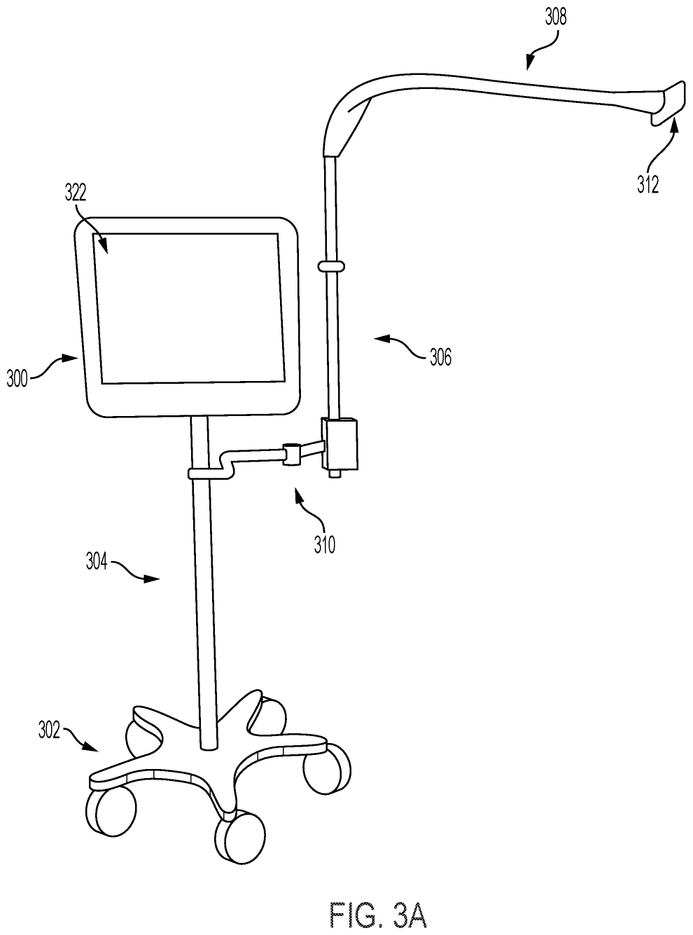

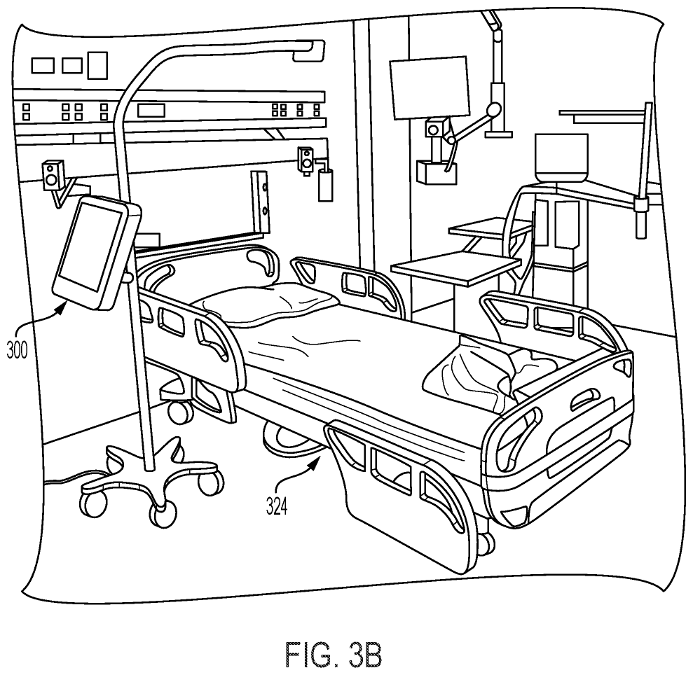

The camera 102 and projector 110 is provided on a support apparatus 108. An example of a support apparatus is described with reference to FIGS. 3A and 3B). The support apparatus 108 supports the camera 102 at a depth sensing position above the patient 102. At the depth sensing position, the camera 102 has a field of view that includes at least part of the bed 106 and at least part of the patient 104 (including the desired region of interest). The support apparatus 108 also supports the projector 110 at a projecting position above the patient 102. At the projecting position, the projector 110 has a field of view that includes at least part of the bed 106 and at least part of the patient 104, including the region of interest.

In the present embodiment, the projection position and depth sensing position are at substantially the position i.e. having the same angle and distance from the patient. However, in alternative embodiments, the depth camera can be provided at a different position than the depth camera and visual feedback from the depth camera may could be used to calibrate and/or adjust the projection from the projector for differences in angle and depth.

In a further embodiment, the projector is manually calibrated for a projection angle and the depth sensing device or processor is configured to correct the sense data to take into account any differences in angle and distance between the projector and camera.

In some embodiments, the depth sensing device is provided at a different distance and angle to each other. In such embodiments, calculations are performed to compensate for any differences in distance and angle so that the projected images spatially correspond to the obtained depth data. In some embodiments, the distance between the depth sensing position and projecting position are sufficiently close that no compensation for the distance between them is required.

In some embodiments, distance between patient and the projection position is taken into account when projecting the image so that the projected image is in focus at the surface of the patient and is also aligned with the patient. Such distance may be due, for example, movement of the patient. In some embodiments, the projector takes into account patient movement due to, for example, breathing. In further embodiments, the projector takes into account the shape of the patient and/or, for example, the bedclothes.

It will be understood that, in the present embodiment, the field of view of the camera 102 and the field of view of the projector 110 may spatially coincide or substantially overlap. In particular, in the case where the fields of view overlap, the overlap between the fields of view will include the region of interest of the patient, so that depth data can be obtained, by the camera 102, from the region of interest, and images based on the processed depth data can be projected by the projector 110 back on to the region of interest.

In the embodiments described herein, the depth data is processed to extract time-varying depth information for a region of interest of the patient, for example, the depth data of a chest region of the patient is processed to obtain time-varying depth information associated with respiration of the patient. However, it will be understood that in some embodiments, the time-varying depth information may be associated with other physiological functions or processes of the patient. As described below, the depth data may also be processed to determine physiological information about the patient.

In some embodiments, physiological information about the patient is extracted by processing the depth data in accordance with known depth data processing techniques. A review of known depth data processing techniques is provided in “Noncontact Respiratory Monitoring Using Depth Sensing Cameras: A Review of Current Literature”, Addison, A. P., Addison, P. S., Smit, P., Jacquel, D., & Borg, U. R. (2021). Sensors, 21(4), 1135. The physiological information may include, for example, information related to respiration, breathing or heart rate, for example, respiration rate, pulse rate, tidal volume, minute volume, effort to breathe, oxygen saturation or any breathing parameter or vital sign. Physiological information may include any parameter or signal associated with the functioning of the body. The physiological information may be obtained from the depth data using known depth data processing techniques.

The image-based depth sensing device may have depth sensor elements that sense light having infra-red wavelengths. The depth sensor elements may sense electromagnetic radiation having wavelengths in the range 1 mm to 700 nm. While an infra-red wavelength depth sensing camera is described, it will be understood that other wavelengths of light or electromagnetic radiation may be used.

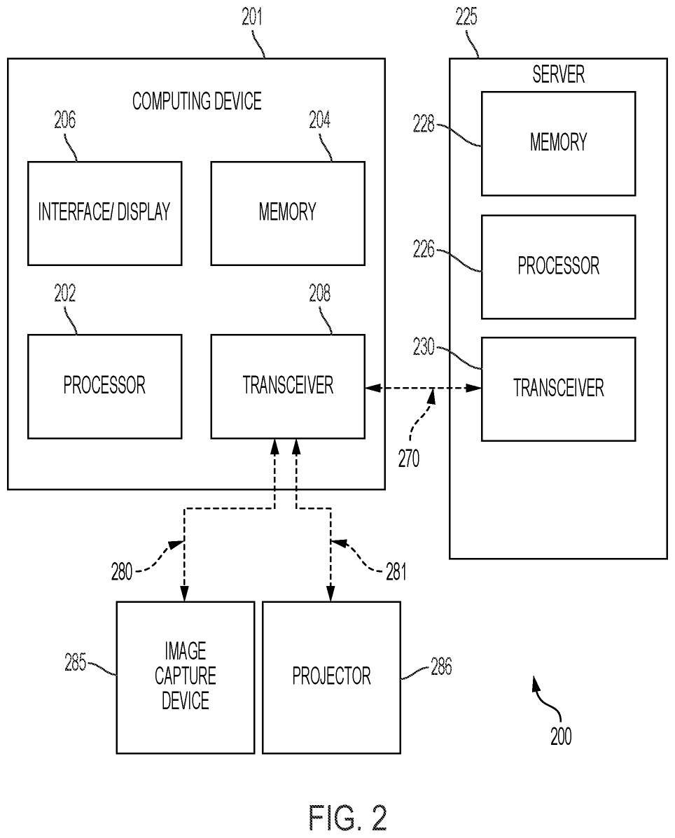

While only a single camera is depicted in FIG. 1, FIG. 2, and FIG. 3, it will be understood that, in some embodiments, multiple cameras and/or multiple projectors may be mounted or positioned about the patient. Depth data obtained from these multiple viewpoints may be combined to obtain further information about the patient. Likewise, the projector may project onto the patient from multiple angles to create a combined projected image.

The field of view of the camera may be defined by a first subtended angle and a second subtended angle. The first and second subtended angles may be in the range, for example, 10 to 100 degrees. In further embodiments, the first and second subtended angles may be in the range 40 to 95 degrees. Likewise, the field of view of the projector may be defined by a first subtended angle and a second subtended angle. The first and second subtended angles may be in the range, for example, 10 to 100 degrees. In further embodiments, the first and second subtended angles may be in the range 40 to 95 degrees. The fields of view of the camera may substantially correspond to the field of view of the projector.

While the camera 102 may be a depth sensing camera, in accordance with various embodiments, any image-based or video-based depth sensing device may be used. For example, a suitable depth sensing device may be a depth sensor that provides depth data for object in the field of view. In some embodiments, the system has an image capture device for capturing images across its field of view together with an associated depth sensor that provides depth data associated with the captured images. The depth information is obtained as a function of position across the field of view of the depth sensing device.

In some embodiments, the depth data can be represented as a depth map or a depth image that includes depth information of the patient from a viewpoint (for example, the position of the image capture device). The depth data may be part of a depth data channel that corresponds to a video feed. The depth data may be provided together with image data that comprises red, green, blue (RGB) data, such that each pixel of the image has a corresponding value for RGB and depth. The depth data may be representative or indicative of a distance from a viewpoint to a surface in the vehicle. This type of image or map can be obtained by a stereo camera, a camera cluster, camera array, or a motion sensor. When multiple depth images are taken over time in a video stream, the video information includes the movement of the points within the image, as they move toward and away from the camera over time.

The captured images, in particular, the image data corresponding to the captured images and the corresponding depth data are sent to a computing device 118 through a wired or wireless connection 114. The computing device 118 includes a processor 120, a display 122, and hardware memory 124 for storing software and computer instructions. Sequential image frames of the occupant are recorded by the camera 102 and sent to the processor 120 for analysis. The display 122 may be remote from the camera 102, such as a video screen positioned separately from the processor and memory.

The processor is further configured to generate projection image data based on the obtained depth data. The generated projection image data is representative of one or more images to be projected by the projector. The generated projection image data is transmitted to the projector 110. The projector 110 is configured to read the projection image data and project the images.

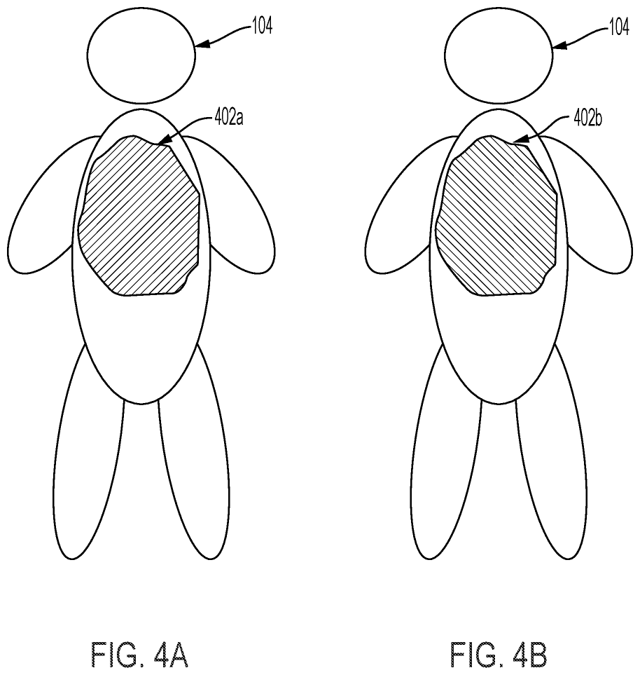

In the present embodiment, the projector 110 is configured to project a sequence of images in real-time. As described with reference to FIGS. 4, 5, and 6, in accordance with embodiments, the projected sequence of images are based on time varying depth information or physiological information obtained by processing the depth data.

Other embodiments of the computing device may have different, fewer, or additional components than shown in FIG. 1. In some embodiments, the computing device may be a server. In other embodiments, the computing device of FIG. 1 may be additionally connected to a server (e.g., as shown in FIG. 2 and discussed below). The depth data associated with the images/video can be processed or analysed at the computing device and/or a server to obtain time-varying depth information for the patient.

FIG. 2 is a block diagram illustrating a patient monitoring system 200, having a computing device 201, a server 225, an image capture device 285 and a projection device 286 according to embodiments. In various embodiments, fewer, additional and/or different components may be used in a system.

The computing device 201 includes a processor 202 that is coupled to a memory 204. The processor 202 can store and recall data and applications in the memory 204, including applications that process information and send commands/signals according to any of the methods disclosed herein. The processor 202 may also display objects, applications, data, etc. on an interface/display 206. The processor 202 may also receive inputs through the interface/display 206. The processor 202 is also coupled to a transceiver 208. With this configuration, the processor 202, and subsequently the computing device 201, can communicate with other devices, such as the server 225 through a connection 270 and the image capture device 285 through a connection 280. Likewise, the processor 202, and subsequently the computing device 201, can communicate with projector 286 through a connection 281. For example, the computing device 201 may send to the server 225 information such as depth information or physiological information of the patient, determined about the occupant by depth data processing.

The computing device 201 may correspond to the computing device of FIG. 1 (computing device 118) and the image capture device 285 may correspond to the image capture device of FIG. 1 (camera 102). Accordingly, the computing device 201 may be located remotely from the image capture device 285 and projector 286, or it may be local and close to the image capture device 285 and projector 286.

In various embodiments disclosed herein, the processor 202 of the computing device 201 may perform the steps described herein. In other embodiments, the steps may be performed on a processor 226 of the server 225. In some embodiments, the various steps and methods disclosed herein may be performed by both of the processors 202 and 226. In some embodiments, certain steps may be performed by the processor 202 while others are performed by the processor 226. In some embodiments, information determined by the processor 202 may be sent to the server 225 for storage and/or further processing.

In some embodiments, the image capture device 285 is or forms part of a remote depth sensing device or depth determining device. The image capture device 285 can be described as local because it is relatively close in proximity to a patient so that at least a part of the patient is within the field of view of the image capture device 285. In some embodiments, the image capture device 285 can be adjustable to ensure that the occupant is captured in the field of view. For example, the image capture device 285 may be physically movable, may have a changeable orientation (such as by rotating or panning), and/or may be capable of changing a focus, zoom, or other characteristic to allow the image capture device 285 to adequately capture the occupant for monitoring. In various embodiments, a region of interest may be adjusted after determining the region of interest. For example, after the ROI is determined, a camera may focus on the ROI, zoom in on the ROI, centre the ROI within a field of view by moving the camera, or otherwise may be adjusted to allow for better and/or more accurate tracking/measurement of the movement of a determined ROI.

In some embodiments, the projection device 286 can be described as local because it is relatively close in proximity to an occupant so that at least a part of the patient is within the field of view of the projector 286. In some embodiments, the projector 286 can be adjustable to ensure that the patient is captured in the field of view. For example, the projector 286 may be physically movable, may have a changeable orientation (such as by rotating or panning), and/or may be capable of changing a focus, zoom, or other characteristic to allow the projector 286 to adequately project images onto the patient.

The server 225 includes a processor 226 that is coupled to a memory 228. The processor 226 can store and recall data and applications in the memory 228. The processor 226 is also coupled to a transceiver 230. With this configuration, the processor 226, and subsequently the server 225, can communicate with other devices, such as the computing device 201 through the connection 270.

The devices shown in the illustrative embodiment may be utilized in various ways. For example, any of the connections 270, 280 and 281 may be varied. Any of the connections 270, 280 and 281 may be a hard-wired connection. A hard-wired connection may involve connecting the devices through a USB (universal serial bus) port, serial port, parallel port, or other type of wired connection that can facilitate the transfer of data and information between a processor of a device and a second processor of a second device. In another embodiment, any of the connections 270, 280 and 281 may be a dock where one device may plug into another device. In other embodiments, any of the connections 270, 280 and 281 may be a wireless connection. These connections may take the form of any sort of wireless connection, including, but not limited to. Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another embodiment, the various devices may connect through an internet (or other network) connection. That is, any of the connections 270, 280 and 281 may represent several different computing devices and network components that allow the various devices to communicate through the internet, either through a hard-wired or wireless connection. Any of the connections 270, 280 and 281 may also be a combination of several modes of connection.

It will be understood that the configuration of the devices in FIG. 2 is merely one physical system on which the disclosed embodiments may be executed. Other configurations of the devices shown may exist to practice the disclosed embodiments. Further, configurations of additional or fewer devices than the ones shown in FIG. 2 may exist to practice the disclosed embodiments. Additionally, the devices shown in FIG. 2 may be combined to allow for fewer devices than shown or separated such that more than the three devices exist in a system. It will be appreciated that many various combinations of computing devices may execute the methods and systems disclosed herein. Examples of such computing devices may include other types of devices and sensors, infrared cameras/detectors, night vision cameras/detectors, other types of cameras, radio frequency transmitters/receivers, smart phones, personal computers, servers, laptop computers, tablets, blackberries, RFID enabled devices, or any combinations of such devices.

FIG. 3A depicts a 3D rendered image of a patient monitoring system 300, in accordance with an embodiment. The system 300 has a support apparatus corresponding to supporting apparatus 108 described with reference to FIG. 1. The support apparatus is mobile and has a moveable base portion 302, a support body 304 and a support arm. The moveable base portion 302 has wheels and is moveable along a floor to position the system 300 adjacent to a bed, for example. The support arm has a first (vertical) extending portion 306 and a second (vertical) extending portion 308. The first extending portion 306 is connected at to an upper end of the support body 302 by a connecting member 310.

At a distal end of the support arm (at the terminal end of the second extending portion 308) there is provided a fitting 312 for a camera and a projector. The support arm is shaped to position the camera and projector at their respective sensing and projecting positions, at a height above the bed. At these positions, the camera is operable to capture depth data about a desired region of the patient and the projector is similarly operable to project images comprising visual indicators on to the patient. FIG. 3A also shows display 322 supported by support body 304. It will be understood that the other elements of the patient monitoring system (for example, the processor) are not depicted in FIG. 3A, for clarity.

FIG. 3B depicts the system 300 in a clinical environment in a bedside adjacent position. FIG. 3B depicts system 300 A configuration of the system 100 and bed 324. As can be observed from FIG. 3B, the support apparatus provides the projector and camera at an elevated position above the bed 324 such that, in use, their respective fields of view include a region of interest of a patient lying in the bed.

In some embodiments, a calibration step is performed, in particular, for some patients, such as neonates, a calibration step may be required. For some patients, for example, adult patients, no calibration is required. In some embodiments, the process is configured to perform an algorithm to determine the subject's depth from the camera and then adjust, for example to optimize, the projected image based on the calibrated depth. In a further embodiment, the depth camera has a fixed focus depth for cases where the patient is not expected to move, and the camera remains at a fixed distance from the patient.

FIGS. 3A and 3B depict the system in one example embodiment. In other embodiments, the projector and camera may be secured in elevated positons using alternative support apparatuses. As a non-limiting example, an arm support secured at a first end to a wall behind a bed supports the camera and project. In a further non-limiting example, a support apparatus secured to part of the bed or a platform attached to the bed (for example, to the back or side of the bed) is used to support the camera and projector. In a further non-limiting example, a support apparatus for supporting the camera and project is secured at a base to the floor. In a further non-limiting example, a support apparatus is secured to a ceiling above a bed to support the camera and projector at the elevated position.

FIGS. 4A and 4B are schematic illustrations of images projected onto a patient 104, in accordance with an embodiment. FIG. 4A depicts a projected image having a visual indicator 402. The image included the visual indicator 402 is projected using projector 110, as described with reference to FIGS. 1 and 2. The visual indicator may be considered as an overlay. It will be understood that the appearance of the projected image and visual indicators have a degree of transparency such that the patient is visible under the visual indicator.

In the present embodiment, the visual indicators provide a projected visualisation of the breathing cycle over time using colour changes. In this embodiment, a first colour is used to represent inhalation (FIG. 4A) and a second colour is used to represent exhalation (FIG. 4B). In some embodiments, a single colour (or other visual property) may be used only to represent inhalation and no colour used for exhalation.

As a first step, a region of interest on the patient is identified using known methods and depth data from the region of interest is obtained. FIG. 4A and FIG. 4B shows a visual indicator (402a, 402b) that spans a projection area that corresponds to the identified region of interest (in this embodiment, the chest region of the patient). In some embodiments, the depth sensing device obtains depth data of the ROI by directing the image capture device toward the ROI and capturing a sequence of two or more images (e.g., a video sequence) of the ROI. FIG. 4A illustrates outward movement (e.g., in real-time) of a patient's torso within the ROI, whereas the FIG. 4B illustrates inward movement (e.g., in real-time) of the patient's torso within the ROI.

Using two images of the two or more captured images, the processor can calculate change(s) in depth over time of the region relative to the depth sensing device. The depth data may represent the distance (in this embodiment, the height) between a region of the patient and the depth sensing device. The depth data is a function of position such that parts (e.g., one or more pixels or groups of pixels) within a ROI have different depths. For example, the system can compute a difference between a first depth of a first part in the ROI 102 in a first image of the two or more captured images and a second depth of the first part in the ROI 102 in a second image of the two or more captured images.

The camera 102 obtains a sequence of depth images that represent depth as a function of position across the image. The depth data of the sequence of images are processed to obtain to obtain time varying depth information for the region of interest. In the present embodiment, the depth information comprises a calculated change in depth (for example, between successive images). The processor is configured to generate projection image data for the projector that is representative of a corresponding sequence of images based on the calculated change in depth. As part of the generation of the projection image data, the processor assigns visual attributes (for example, colour, shade, pattern, concentration and/or intensity) to one or more parts of the visual indicator based on the calculated change of depth such that the visual indicator, projected onto the patient, has an appearance dependent on the calculated change in depth. In this embodiment, the change of depth varies over time during each breathing cycle.

In the present embodiment, the projection image data represents images having a visual indicator with an appearance based on the calculated change in depth. In particular, FIG. 4 shows the visual indicator 402a having a first colour (for example, green) when the calculated change in depth is negative which corresponds to movement of the surface towards the camera (i.e. the patient 104 inhaling). FIG. 4B shows the visual indicator 402b having a second colour (red) when the calculated change in depth is positive which corresponds to movement of the surface away from the camera (i.e. the patient 104 exhaling). FIG. 4A and FIG. 4B represent the different colours using different shading patterns. It will be understood that, in addition or as an alternative to colour, the appearance of the one or more projected visual indicators may include one or more of shade, pattern, concentration and/or intensity.

The shape (e.g. the outer boundary) of the visual indicator may also change over time, for example, during a breathing cycle. For example, in some embodiments, at certain times of the breathing cycle only parts of the region of interest may record a substantial change in depth. Parts of the region of interest that do not record a substantial change in depth may have no colour, thus the coloured part of the projected visual indicator may change shape through the breathing cycle. It will be further understood, that in the described embodiments, the projected image is transparent except at the region defined by the visual indicators.

While the assigned colour of FIG. 4 depends on the direction of the change of depth, in further embodiments, the appearance of the visual indicator may be dependent on, for example, the magnitude of the change of depth at points of the region of interest. In such embodiments, a concentration or intensity may be selected to correlate with the magnitude of the computed change (for example, so that a first point or region experiencing greater change than a second point or region has a higher concentration or intensity than the second point or region.

The appearance of the visual indicators may have one or more variable characteristics.

In some embodiments, the visual indicators may be applied at a pixel by pixel level such that a visual indication of the depth information for each pixel is projected on to a corresponding part of the patient. In some embodiments, the system can assign visual indicators (e.g., colours, patterns, shades, concentrations, intensities, etc.) from a predetermined visual scheme to regions in an ROI. The visual indicators can correspond to changes in depth of computed by the system (e.g., to the signs and/or magnitudes of computed changes in depth)

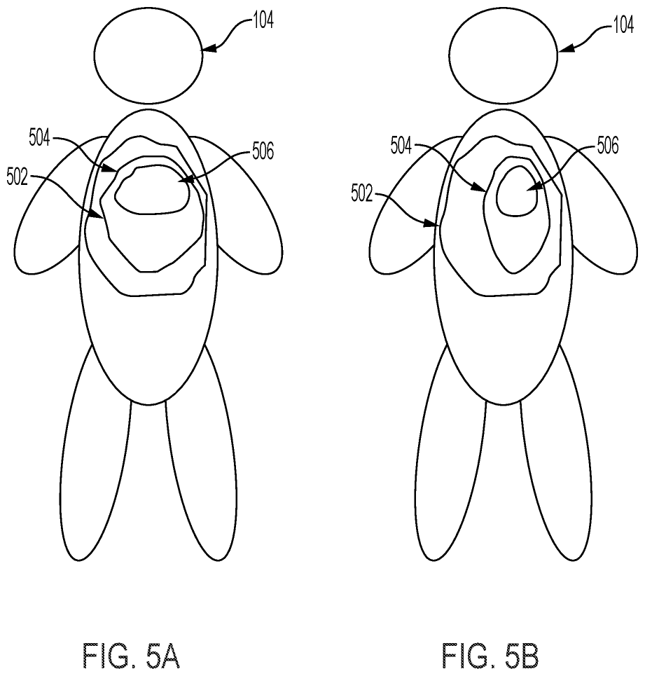

In the embodiment of FIG. 4, the appearance of the visual indicator was dependent on a calculated change of depth corresponding to breathing. FIG. 5A and FIG. 5B illustrate projection of more than one visual indicator onto a patient, in accordance with an embodiment.

In this embodiment, displacement of parts of the patient is displayed using the visual indicators. In this embodiment, differences in displacement between different parts of the chest are visible (for example, a difference in displacement between a first side of the chest and a second side of the chest is displayed). As a non-limiting example, a collapsed lung would result in a first side having a greater measured displacement than the second side and thus the appearance of the projected image at the first side is different to the appearance of the projected image at the second side.

Other quantities relating to breathing may be depicted and represented by the appearance of the visual indicators (such as average breathing rate). In some embodiments, tidal volume (the overall volume change over a breathing cycle) may be displayed using the visual indicators. For example, the colour displayed may represent the tidal volume for the previous breathing cycle.

FIG. 5A depicts three visual indicators projected onto the patient 104. In this embodiment, the three visual indicators are in the form of displacement contours projected onto the patient. The displacement contours correspond to displacement of regions of the patient. In this embodiment, three displacement contours are displayed corresponding to first, second and third displacement ranges. In the present embodiment, the displacement ranges are defined with reference to the minimum and maximum determined displacements in the region of interest. In some embodiments, the displacement contours correspond to ranges of displacement. These could be, for example, the minimum value to the maximum value. Alternatively, these could be set at pre-determined displacement values (deltas) or correspond to pre-determined displacement ranges. As a non-limiting example, the contours could correspond to displacements of: 1 mm, 2 mm, 5 mm, 10 mm. Displacement will be understood to be the change in position over time. The displacement in the present embodiments, is the change in distance from the depth camera to the object in the field of view (in this example, the patient).

FIG. 5A depicts a first contour 502, a second contour 504 and a third contour 506. The area defined by each contour (referred to as the contour area) has a distinct visual property relative to the other contours. In the present embodiment, the visual property is colour: the first area 402 defined by the first contour is green; the second area 404 defined by the second contour is yellow; and the third area 406 defined by the third contour is red. As the first area is larger than the second area, only part of the first area that the second area does not overlap is visible. Likewise, as the third area is smaller than the second area, only part of the second area that does not overlap the third area is visible. While distinct colours are described it will be understood that grading or different hues of colours may be used for different regions.

In the present embodiment, the first area corresponds to a first displacement range, the second area corresponds to a second displacement range and the third area corresponds to a third displacement range.

The definition of the contour lines and the visual property of each contour area is based on the obtained depth information for the region of the patient inside that contour.

In the present embodiment, a predefined region of the patient is identified and depth data from that pre-defined region is processed. The pre-defined region defines a boundary for a region of interest. In the region of interest, more than one contour is defined. The contours can be defined and projected using different methods. In the above-described embodiment, three contour levels are defined, however, it will be understood that the contour steps and number of contour may be determined in accordance with a number of different methods. In some embodiments, a pre-determined number of contour levels are defined and projected. The pre-determined number may be, for example, between 3 and 10. In some embodiments, the number of contour levels may be selected automatically depending on, for example, the total difference between a minimum and a maximum value.

In some embodiments, the projection highlight physiological information. As a non-limiting example, the projected visual indicator indicates that a diaphragm moves in a non-symmetric manner (e.g., left lung is inflating but the right lung is not). In some embodiments, the projected quantities may include, for example, maximum, minimum and indicative tidal volume.

In a further embodiment, the appearance of the visual indicator is dependent on a total displacement of a region of the subject over a breathing cycle. In this embodiment, the total displacement may vary between breathing cycles. In some embodiments the appearance is representative of an accumulated displacement over the breathing cycle.

In the present embodiment, the largest contour boundary corresponds to an identified region of interest and the other contours are drawn based on values of sensed displacement. In the present embodiment, the contours represent displacement information, in particular, a calculated total displacement over a breathing cycle. The appearance of each region is therefore dependent on the degree of movement over a breathing cycle: the first region moves a lesser distance over the breathing cycle than the second region and the second region moves a lesser distance over the breathing cycle than the third region. This division into three groups can be performed by processing the determined displacement values and grouping the values into pre-determined ranges. For example, three groups (low, medium and high) total displacement may be defined and the regions defined accordingly.

FIG. 5B depicts visual indicators substantially as described with reference to FIG. 5A. However, in FIG. 5B, the second and third regions are substantially smaller and projected only one side of the patient's body. As depicted in FIG. 5B, the asymmetry in breathing may therefore be easily recognised when using the monitoring system.

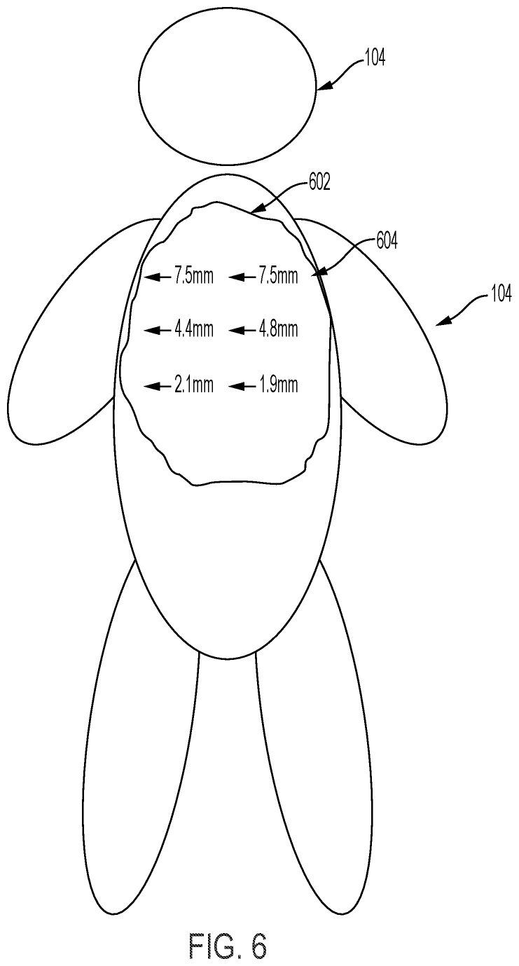

FIG. 6 depicts further visual indicators projected on the patient that include numerical data, in accordance with a further embodiment. In this embodiment, in addition to the first visual indicator 602 corresponding to the region of interest (which may correspond to the visual indicator described with reference to FIG. 4), six further visual indicators 604 are projected onto the patient 104. Each further visual indicator has numerical data corresponding to a value of total displacement over a breathing cycle. That numerical data is projected onto a number of different positions on the patient. Each further visual indicator also includes an arrow pointing to the measurement location. The total displacement determined using depth data will vary across the region of interest and therefore the values of the numerical data will also vary according the region of interest. The total displacement can be measured as the difference between the maximum depth value and the minimum depth value over the breathing cycle.

In further embodiments, velocity information of the monitored region may also be determined and the appearance of the visual indicator may be based on the velocity information. In this embodiment, the velocity of a point in the scene is the local change in distance between the camera and the point in the scene. The velocity can be computed, for example, by monitoring the depth changes in a local region over a time step. The time step can be selected as the time between frames and the velocity may be determined and updated at every frame.

The system can also project substantially no visual indicator to regions that are determined to neither move away or away from the depth sensing device over time. For example, such regions may show substantially negligible changes in depth or changes in depth equivalent to zero across two or more images, no colour is projected on to the region.

In further embodiments, the appearance of one or more visual indicators is based on a physiological parameter determined from the time-varying depth information. For example, a respiration rate is determined and the colour of a projected region on the patient is based on the value of the determined respiration rate. In such an embodiment, the projected region may have a first colour (e.g. blue) for a respiration rate corresponding to a slow respiration rate, a second colour (e.g. green) for a respiration rate corresponding to a normal respiration rate and a third colour (e.g. red) for a respiration rate corresponding to a fast respiration rate.

In some embodiments, the system may assign a new pattern or no pattern to regions that exhibit changes in depth that the system determines are not physiological and/or are not related to respiratory motion (for example, changes in depth that are too quick, changes in depth indicative of gross body movement etc.).

Regardless of the visual scheme employed, the system can project (for example, in real time) the visual indicators to parts of the patient corresponding to the ROI. Thus, the projected visual indicators may emphasize subtle changes in depths detected by the system. In turn, a user (e.g., a caregiver, a clinician, a patient, etc.) can quickly and easily determine whether or not a patient is breathing based on whether or not visual indicators corresponding to one or more breathing cycles of the patient are displayed over the ROI on the patient. This may help a user and/or a video-based patient monitoring system to detect a variety of medical conditions, such as apnea, rapid breathing (tachypnea), slow breathing, intermittent or irregular breathing, shallow breathing, and others.

In addition, the system may allow breathing to be observed without the need for an observer to get very close to patient. The proximity of the observer to the patient may also trigger an awareness response that could change breathing, and this may be avoided using the system described above. By including a topographical display overlaid on the patient, eye contact may be maintained and the need for a clinician to look at a separate display may be avoided. Further benefits may include, for example, easy observation of paradoxical breathing and/or detecting regions of the chest that are moving to a lesser degree than other regions.

In the above-described embodiments, regions of interest (ROIs) are described. Known methods for defining regions of interest (ROIs) on a patient can be used. For example, the system can define a ROI using a variety of methods (e.g., using extrapolation from a point on the patient, using inferred positioning from proportional and/or spatial relationships with the patient's face, using parts of the patient having similar depths from the camera as a point, using one or more features on the patient's clothing, using user input, etc.). In some embodiments, the system defines an aggregate ROI that, for example, includes both sides of the patient's chest as well as both sides of the patient's abdomen. An aggregate ROI can be useful in determining a patient's aggregate tidal volume, minute volume, and/or respiratory rate, among other aggregate breathing parameters. In these and other embodiments, the system can define one or more smaller regions of interest within the patient's torso. In these and other embodiments, the system can define other regions of interest (for example, regions of interest corresponding to: patient's chest; patient's abdomen; right and/or left side of patient's chest or torso. The system can define one or more other regions of interest, for example, the system can define a region of interest that includes other parts of the patient's body, such as at least a portion of the patient's neck (e.g., to detect when the patient is straining to breathe).

The systems and methods described herein may be provided in the form of tangible and non-transitory machine-readable medium or media (such as a hard disk drive, hardware memory, etc.) having instructions recorded thereon for execution by a processor or computer. The set of instructions may include various commands that instruct the computer or processor to perform specific operations, such as the methods and processes of the various embodiments described herein. The set of instructions may be in the form of a software program or application. The computer storage media may include volatile and non-volatile media, and removable and non-removable media, for storage of information such as computer-readable instructions, data structures, program modules or other data. The computer storage media may include, but are not limited to, RAM, ROM, EPROM, EEPROM, flash memory or other solid-state memory technology, CD-ROM, DVD, or other optical storage, magnetic disk storage, or any other hardware medium which may be used to store desired information and that may be accessed by components of the system. Components of the system may communicate with each other via wired or wireless communication. The components may be separate from each other, or various combinations of components may be integrated together into a medical monitor or processor, or contained within a workstation with standard computer hardware (for example, processors, circuitry, logic circuits, memory, and the like). The system may include processing devices such as microprocessors, microcontrollers, integrated circuits, control units, storage media, and other hardware.

A skilled person will appreciate that variations of the enclosed arrangement are possible without departing from the invention.

For example, the above described embodiments, describe the appearance of one or more visual indicators based on determined time varying depth information. In further embodiments, the depth data may be processed using known depth data processing methods to obtain physiological information, for example, physiological parameters, and the appearance of the visual indicators may be based on said physiological parameters. In a non-limiting example, a respiration rate calculated by processing depth data may be used to change the colour of projected visual indicator (for example, a slow rate may be coloured blue, a normal rate may be colour green and a fast rate may be colour red).

As a further example, while the above-described embodiments refer to a system for monitoring a patient lying in a bed, it will be understood that the system may be configured to operate for a patient in a different position. In addition, for respiration information or other physiological signals, the camera and/or projector may be provided at a different viewpoint.

As a further example, as described above, depth data may be processed to determine a physiological signal. In further embodiments, noise information associated with the determined signal or the obtained depth data itself, for example, due to the body motion, is determined and a visual indicator representing the noise is projected onto the body. The presence of noise, for example, due to movement may mean that the determined signal and hence projected visual indicator is not reliable at that time. In some embodiments, the presence of noise is indicated by projecting a visual indicator having a selected colour or adjusting the projected visual indicator to indicate the presence of noise (by adjusting one or more of shape, colour, shade, pattern, concentration and/or intensity). In some embodiments, the visual indicator has a visual aspect representing the level of noise. In some embodiments, alternatively or in addition, a noise presence message is projected onto the scene in response to determining an unacceptable level of noise in the signal. For example, as a non-limiting example, the noise presence message “Invalid Signal” or “Possible Motion” may be projected on to the scene. The presence of noise will be understood, in some embodiments, to be a level of noise about a pre-determined threshold.

Accordingly, the above description of the specific embodiment is made by way of example only and not for the purposes of limitations. It will be clear to the skilled person that minor modifications may be made without significant changes to the operation described.

1. A system comprising:
at least one depth determining device configured to determine depth data representing depth across a field of view;
a processor configured to process the depth data to obtain at least one of time varying depth or physiological information associated with at least one of respiration or another physiological function; and
a projector configured to project one or more images into the field of view, wherein at least part of the one or more images is based on at least one of the obtained time varying depth or the physiological information.

2. The system of claim 1, wherein the depth data is sensed from a region of interest of a subject thereby to obtain time varying depth or physiological information associated with respiration or another physiological function of the subject and wherein the image is projected back on to the region of interest of the subject.

3. The system of claim 2, wherein the projected one or more images are in spatial correspondence with the obtained time varying depth or physiological information.

4. The system of claim 1, wherein the time varying depth or physiological information comprises a sign and/or a magnitude of a calculated change in depth.

5. The system of claim 1, wherein the time varying depth or physiological information comprises a physiological parameter.

6. The system of claim 5, wherein the physiological parameter comprises at least one of: respiration rate, pulse rate, tidal volume, minute volume, effort to breathe, oxygen saturation.

7. The system of claim 2, wherein the time varying depth or physiological information comprises a total displacement or velocity of a region of the subject over a breathing cycle.

8. The system of claim 2, wherein the time varying depth or physiological information comprises a magnitude and/or sign of movement relative to the depth determining device.

9. The system of claim 2, wherein the one or more projected images comprises one or more visual indicators having an appearance based at least in part on the determined time varying depth or physiological information.

10. The system of claim 9, wherein the appearance of the one or more visual indicators comprises at least one of: a colour, shade, pattern, concentration and/or intensity.

11. The system of claim 9, wherein the depth data are obtained for a region of interest of a subject and the visual indicator of the one or more projected images may substantially span the region of interest.

12. The system of claim 9, wherein the one or more visual indicators comprises at least one of: an overlay, a boundary and/or an area defining a region, textual or numerical data, an arrow, one or more contours.

13. The system of claim 9, wherein the one or more visual indicators comprise two or more visual indicators corresponding to two or more regions, wherein the appearance of each of the two or more visual indicators is based on the time varying depth information or physiological information obtained for that region.

14. The system of claim 9, wherein the two of more images comprises a sequence of moving images and are projected in real time such that the projected images change in response to the changes in the time varying depth or physiological information.

15. The system of claim 9, wherein the one or more visual indicators has a first appearance when the determined time varying depth information or physiological information is indicative of movement of the region away from the depth determining device and a second appearance when the determined time varying depth information corresponds to movement of the region toward the depth determining device.

16. The system of claim 9, wherein the one or more visual indicators comprises a further appearance when the determined time varying depth information is indicative of lack of movement of the region relative to the depth determining device.

17. The system of claim 9, wherein the processor is configured to determine noise information associate with the time varying depth or physiological information and wherein the one or more images is based on the determined noise information.

18. The system of claim 9, wherein the projector is an optical projector configured to generate and project an optical image and/or wherein the at least one depth determining device comprises at least one of: a depth sensing camera, a stereo camera, a camera cluster, a camera array, a motion sensor.

19. A method comprising:
determining depth data representing depth across a field of view;
processing the depth data to obtain time varying depth or physiological information associated with respiration and/or another physiological function; and
projecting one or more images into the field of view, wherein at least part of the one or more images is based on the obtained time varying depth or physiological information.

20. A non-transitory computer readable medium comprising instructions operable by a processor to:
receive depth data representing depth across a field of view,
process depth data to obtain time varying depth or physiological information associated with respiration and/or another physiological function; and
generate projection image data representative of one or more images for projecting into the field of view, wherein at least part of the one or more images is based on the obtained time varying depth or physiological information.
